(19) United States
(12) Patent Application Publication
(10) Pub. No.: US 2012/0310174 A1
(43) Pub. Date: Dec. 6, 2012

(54) PORTABLE ENEMA DEVICE

(76) Inventor: Byung Eun Yoo, Daegu (KR)

(21) Appl. No.: 13/513,561

(22) PCT Filed: Feb. 26, 2010
(86) PCT No.: PCT/KR2010/001232

§ 371 Date: Jun. 1, 2012

(51) Int. Cl. A61M 3/02

(52) U.S. Cl. 604/212

(57) ABSTRACT

The portable enema device of the present invention can be used by patients experiencing bowel trouble such as constipation and is especially convenient for individuals to carry and use at any time. The portable enema device includes: an insertion nozzle; a tube-type manual pump; a supply hose; an upper cap which is detachably fastened to an upper end of the tube-type manual pump in a screw coupling manner; an inner supply tube which extends into the tube-type manual pump; a sliding pipe which is formed with an infusion solution supply path and is placed under the tube-type manual pump; and an inner sliding pipe provided in the shape of a concentric circle.

### TECHNICAL FIELD

The present invention relates to a portable enema device which can be used by patients experiencing bowel trouble such as constipation, and more particularly to a portable enema device convenient for individuals to carry and use at any time.

### BACKGROUND ART

As well known in the art, enema devices have been widely used by patients experiencing troubles with stool or colon disorders such as constipation, since the enema devices can remove toxins due to feces contained long in the colon and provide an effect of preventing and treating various diseases.

Although various types of enema devices have been proposed, conventional enema devices are inconvenient due to their complicated structures making it difficult to use the devices and inefficient operation when a user supplies an infusion solution to the colon through the anus.

### DISCLOSURE

### Technical Problem

The present invention is directed to solving the problems of the related art as described above, and an aspect of the present invention is to provide a convenient portable enema device for patients or users.

### Technical Solution

In accordance with an aspect of the present invention, a portable enema device includes an insertion nozzle through which an infusion solution is supplied to the anus; a tube-type manual pump supplying the infusion solution to the insertion nozzle; and a supply hose supplying the infusion solution from outside into the tube-type manual pump, the improvement including: an upper cap integrally provided with the insertion nozzle and detachably fastened to an upper end of the tube-type manual pump in a screw coupling manner; an inner supply tube, which is in communication with the insertion nozzle, includes an upper check valve and extends into the tube-type manual pump; and a sliding pipe including an infusion solution supply path and disposed under the tube-type manual pump to be inserted into or drawn out of the tube-type manual pump. Here, the sliding pipe includes an inner sliding pipe concentrically disposed therein to be inserted into or drawn out of the sliding pipe in a longitudinal direction thereof.

### Advantageous Effects

As described above, a portable enema device according to the present invention has a simple structure, is easy to use, and convenient for a user to carry, store and use at any time.

### DESCRIPTION OF DRAWING

FIG. 1 is a perspective view of an enema device according to the present invention;

FIG. 2 is a cross-sectional view taken along line I-I of FIG. 1;

FIG. 3 is a perspective view of the enema device, from which a sliding pipe and an inner sliding pipe protrude, according to the present invention;

FIG. 4 is a cross-sectional view taken along line II-II of FIG. 3; and

FIGS. 5(A) to 5(D) are flowcharts showing operation of the enema device according to the present invention.

### BEST MODE

Next, exemplary embodiments of the present invention will be described in detail with reference to accompanying drawings.

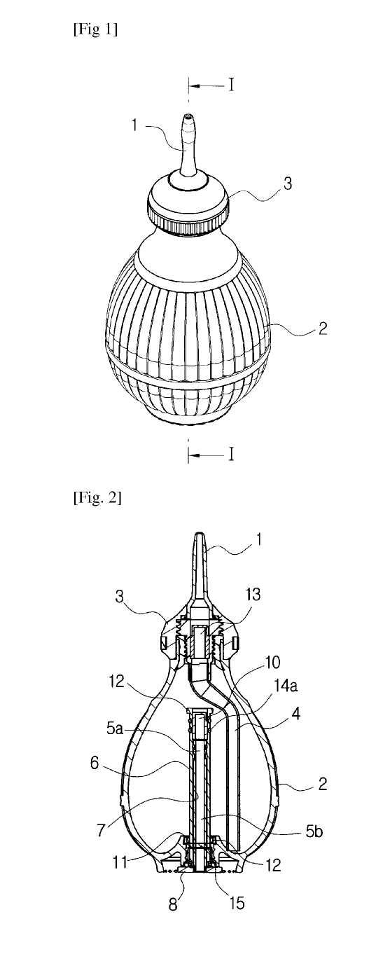

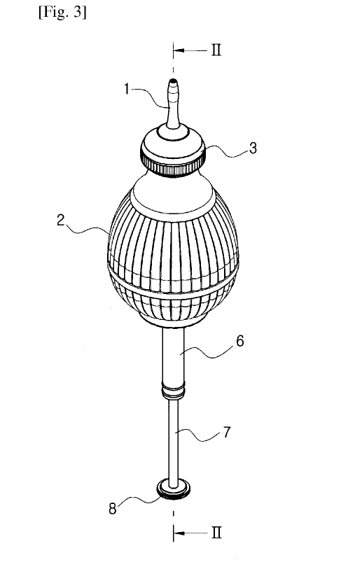

FIG. 1 is a perspective view of an enema device according to the present invention; FIG. 2 is a cross-sectional view taken along line I-I of FIG. 1; FIG. 3 is a perspective view of the enema device, from which a sliding pipe and an inner sliding pipe protrude, according to the present invention; and FIG. 4 is a cross-sectional view taken along line II-II of FIG. 3.

The present invention relates to an enema device, which may be used by patients experiencing bowel trouble such as constipation. The enema device includes an insertion nozzle 1 through which an infusion solution is supplied to the anus; a tube-type manual pump 2 which supplies the infusion solution to the insertion nozzle 1; and a supply hose which supplies the infusion solution from outside into the tube-type manual pump 2, in which an upper cap integrally provided with the insertion nozzle is detachably fastened to an upper end of the tube-type manual pump 2 in a screw coupling manner; an inner supply tube 4 communicating with the insertion nozzle 1 and having an upper check valve 13 extends into the tube-type manual pump 2; a sliding pipe 6 having a infusion solution supply path 5a is placed under the tube-type manual pump 2 to be inserted into or drawn out of the tube-type manual pump 2; and an inner sliding pipe 7 is concentrically disposed in the sliding pipe to be inserted into or drawn out of the sliding pipe in a longitudinal direction thereof.

Here, the inner sliding pipe 7 longitudinally dispose in the sliding pipe 6 is formed with another infusion solution supply path 5b communicating with the infusion solution supply path 5a of the sliding pipe 6 and is integrally formed at a distal end thereof with a nut-type gripper 8.

In addition, the inner sliding pipe 7 is provided at a leading end thereof with an O-ring 14a to prevent the inner sliding pipe 7 from being completely drawn out of the sliding pipe 6 when the inner sliding pipe 7 is drawn out of the sliding pipe 6.

Further, the sliding pipe 6 is provided at a lower leading end thereof with a stopper 9 such that the sliding pipe 6 is caught by a holder 15 and prevented from entering the tube-type manual pump 2 when the sliding pipe 6 is pushed into the tube-type manual pump 2 after using the enema device.

The sliding pipe 6 is provided at an upper end thereof with a lower check valve 10 to prevent an infusion solution, which has been supplied to the tube-type manual pump 2 through the other infusion solution supply path 5b of the inner sliding pipe 7, from leaking through the infusion solution supply path 5a of the sliding pipe 6 and the infusion solution supply path 5b of the inner sliding pipe 7.

The holder 15 is placed in a lower inlet 11 of the tube-type manual pump 2 to be disposed within or under the tube-type manual pump 2, such that the sliding pipe 6 is fitted into the holder 15. Here, the sliding pipe 6 is integrally formed at an upper end thereof with an upper projection 12 having on O-ring 14b, which acts as a packing member and is fitted into a hole of the holder 15 in the lower inlet 11 of the tube-type manual pump 2 to prevent the infusion solution in the tube-type manual pump 2 from leaking out when the sliding pipe 6 is drawn out.

Since the enema device according to this embodiment is applied to the anus, the insertion nozzle 1 and the tube-type manual pump 2 are made of a silicon material harmless to humans.

Now, operation of the portable enema device will be described.

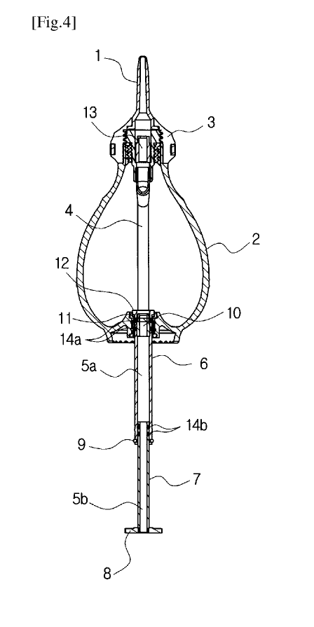

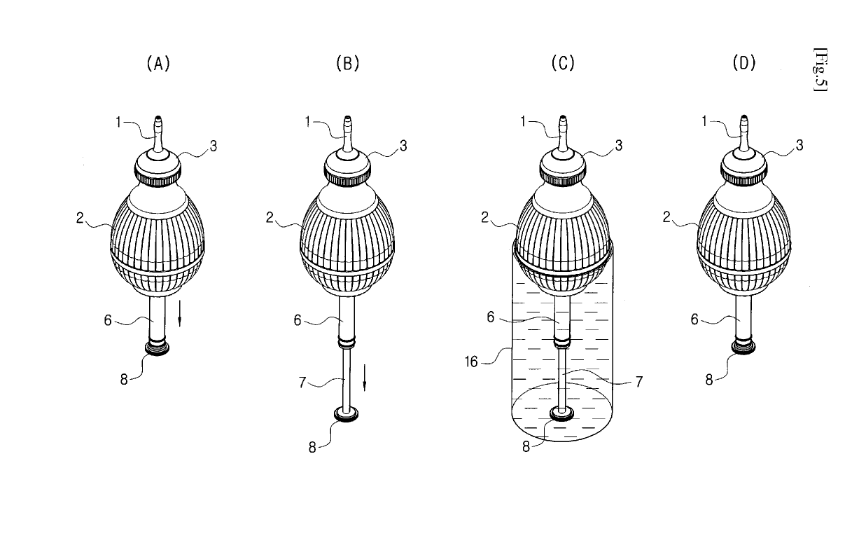

When a user pulls a nut-type grip 8 with his/her hand from the portable enema device as shown in FIGS. 1 and 2, the sliding pipe 6 is first drawn out. At this time, the upper projection 12 of the sliding pipe 6 is seated on the holder 15 in the lower inlet 11 of the tube-type manual pump 2 [see FIG. 5(A)].

Then, when the nut-type grip 8 is further pulled out, the inner sliding pipe 7 is drawn out of the sliding pipe 6, as shown in FIG. 5(B) or FIG. 4.

Then, the sliding pipe 6 and the inner sliding pipe 7 are immersed in the case 16 filled with a clean infusion solution (such as distilled water), with both the inner sliding pipe 7 and the sliding pipe 6 drawn out as shown in FIG. 5(C), and the tube-type manual pump 2 is operated by hand to supply the infusion solution to the tube-type manual pump 2 through the infusion solution supply paths 5b, 5a until the tube-type manual pump 2 is filled with a desired amount of the infusion solution to be stored therein.

Then, with the inner sliding pipe 7 inserted into the sliding pipe 6 as shown in FIG. 5(D), a user inserts the insertion nozzle 1 into the anus and operates the tube-type manual pump 2 to supply the infusion solution from the tube-type manual pump 2 to the colon through the insertion nozzle 1 via the inner supply tube 4. At this time, the upper check valve 13 of the inner supply tube 4 prevents the infusion solution supplied through the insertion nozzle 1 from backward flowing into the tube-type manual pump 2.

When the infusion solution remains in the tube-type manual pump 2 after using the device, a pumping operation is performed to remove the infusion solution from the tube-type manual pump 2. Then, a user separates the upper cap 3 including the insertion nozzle 1 from the tube-type manual pump 2 to clean the upper cap 3 for subsequent use.

1. A portable enema device including an insertion nozzle through which an infusion solution is supplied to the anus; a tube-type manual pump supplying the infusion solution to the insertion nozzle; and a supply hose supplying the infusion solution from outside into the tube-type manual pump, the improvement comprising: an upper cap integrally provided with the insertion nozzle and detachably fastened to an upper end of the tube-type manual pump in a screw coupling manner; an inner supply tube, which is in communication with the insertion nozzle, includes an upper check valve and extends into the tube-type manual pump; a sliding pipe including an infusion solution supply path and disposed under the tube-type manual pump to be inserted in or drawn out of the interior of the tube-type manual pump; and an inner sliding pipe concentrically disposed in the sliding pipe to be inserted into or drawn out of the sliding pipe in a longitudinal direction thereof.

2. The portable enema device of claim 1, wherein the inner sliding pipe disposed in the longitudinal direction of the sliding pipe is formed with another infusion solution supply path communicating with the infusion solution supply path of the sliding pipe, and is integrally formed at a distal end thereof with a nut-type grip.

3. The portable enema device of claim 1, wherein the inner sliding pipe is provided at a leading end thereof with an O-ring to prevent the inner sliding pipe from being completely drawn out of the sliding pipe when the inner sliding pipe is drawn out of the sliding pipe.

4. The portable enema device of claim 1, wherein the sliding pipe is integrally formed at an upper end thereof with an upper projection having on O-ring, the O-ring acting as a packing member and being fitted into a hole of a holder disposed in a lower inlet of the tube-type manual pump.

5. The portable enema device of claim 1, wherein the inner supply tube and the sliding pipe are provided with check valves, respectively.
